(19) United States
(12) Patent Application Publication
(10) Pub. No.: US 2020/0289601 A1
(43) Pub. Date: Sep. 17, 2020

(54) HEMORRHOID TREATMENT SYSTEMS AND METHOD

(71) Applicant: Marina Laklakian, Sodertalje (SE)

(72) Inventor: Marina Laklakian, Sodertalje (SE)

(21) Appl. No.: 16/732,199

(22) Filed: Dec. 31, 2019

Related U.S. Application Data
(60) Provisional application No. 62/817,616, filed on Mar. 13, 2019.

(51) Int. Cl. A61K 36/88; A61M 35/00; A61K 9/06; A61K 35/54; A61K 35/20

(52) U.S. Cl. CPC A61K 36/88; A61M 35/003; A61K 35/20; A61K 35/54; A61K 9/06

(57) ABSTRACT

A cream made from natural ingredients designed to alleviate pain and discomfort for those that suffer from hemorrhoids. The cream potentially effectively eliminates the need to use chemical-based ointments, and may prevent individuals from needing surgery in order to treat hemorrhoids. Effectively, the cream reduces the amount of recovery time needed to heal, offering a fast and effective method for treating hemorrhoids without the use of chemicals or surgery.

### CROSS-REFERENCE TO RELATED APPLICATION(S)

The present application is related to and claims priority to U.S. Provisional Patent Application No. 62/817,616 filed Mar. 13, 2019, which is incorporated by reference herein in its entirety.

### BACKGROUND OF THE INVENTION

The following includes information that may be useful in understanding the present disclosure. It is not an admission that any of the information provided is prior art nor material to the presently described or claimed inventions, nor that any publication or document that is specifically or implicitly referenced is prior art.

### TECHNICAL FIELD

The present invention relates generally to the field of hemorrhoid treating means of existing art and more specifically relates to drug, bio-affecting and body treating compositions.

### RELATED ART

Many individuals suffer from hemorrhoids, which can cause extreme pain and discomfort. Traditional treatment options may include chemical-based ointments or surgical intervention. Neither of these options are desirable for most individuals. A suitable organic solution is desired.

U.S. Pub. No. 2010/0247689 to Valentina Paspaleeva-Kühn relates to an agent containing fat (oil), which contains onion extract, the production and use thereof for caring, preventing or treating damaged skin tissue, especially scarred tissue. The described agent is a novel agent containing fat (oil), which contains onion extract, in addition to the production and use thereof for caring, preventing or treating damaged skin tissue, such as after operations, biopsies, wounds, burns and other accidents, and especially scarred tissue, stretch marks, degenerative skin conditions and others. The agent is characterized in that the base thereof is based on oil, which can be used to produce, for example, cream, lotion, fluid, massage, or balm based products which contain fail (oil). Surprisingly, the onion extract, which contains water, alcohol or water-alcohol, can be integrated into a base containing oil without the effectiveness thereof being reduced or a phase separation being produced. The effectiveness thereof could be proved when the medical application thereof was monitored.

### SUMMARY OF THE INVENTION

In view of the foregoing disadvantages inherent in the known drug, bio-affecting and body treating compositions art, the present disclosure provides a novel hemorrhoid treatment system. The general purpose of the present disclosure, which will be described subsequently in greater detail, is to provide an effective organic hemorrhoid treatment system.

A method of hemorrhoid treatment with a hemorrhoid treatment system is disclosed, the method comprising the steps at least of providing a mixture of ingredients consisting of equal portions of egg; butter; and onion; and blending the mixture to form a cream.

A hemorrhoid treatment system to treat hemorrhoids is also disclosed, the system comprising a mixture of ingredients formed as a cream consisting essentially of equal portions of egg; butter; and onion; and a substantially air-tight container (substantially air-tight when in a closed-state to avoid degradation of the product) that when in an open-state is suitable for removing (squeezing from a tube or by hand from an openable container) the mixture in a cream form for application to hemorrhoids; the cream able to be applied once dispensed from the substantially air-tight container. The mixture of ingredients is organic consisting of only organic ingredients and does not comprise dye(s), perfume(s), preservative(s), and stabilizer(s). A kit is also disclosed.

For purposes of summarizing the invention, certain aspects, advantages, and novel features of the invention have been described. Not necessarily all such advantages may be achieved in accordance with any one particular embodiment of the invention. Thus, the invention may be embodied or carried out in a manner that achieves or optimizes one advantage or group of advantages as taught without necessarily achieving other advantages as may be taught or suggested. The features of the invention which are believed to be novel are particularly pointed out and distinctly claimed in the concluding portion of the specification. These and other features, aspects, and advantages of the present invention will become better understood with reference to the following drawings and detailed description.

### BRIEF DESCRIPTION OF THE DRAWINGS

The figures which accompany the written portion of this specification illustrate embodiments and methods of use for the present disclosure, a hemorrhoid treatment system, constructed and operative according to the teachings of the present disclosure.

FIG. 1 is a perspective view of the hemorrhoid treatment system during an ‘in-use’ condition; the container comprising a squeeze tube (cap not shown) able to squeeze-dispense the cream according to an embodiment of the disclosure.

FIG. 2 is a perspective view of the hemorrhoid treatment system of FIG. 1, able to be hand-dispensed from a container having a lid (not shown) according to an embodiment of the present disclosure.

FIG. 3 is a view of the hemorrhoid treatment system indicating ingredients used of FIG. 1, according to an embodiment of the present disclosure.

FIG. 4 is a view of the hemorrhoid treatment system of FIG. 1, according to embodiments of the present disclosure.

FIG. 5 is another view of the hemorrhoid treatment system of FIG. 1, according to embodiments of the present disclosure.

The various embodiments of the present invention will be described in conjunction with the appended drawings, wherein like designations denote like elements.

### DETAILED DESCRIPTION

As discussed above, embodiments of the present disclosure relate to drug, bio-affecting and body treating compositions and more particularly to a hemorrhoid treatment system as used to improve the treatment of hemorrhoids.

Generally, hemorrhoid treatment systems is a means treating hemorrhoids made from natural (organic) ingredients. The cream is preferably made from equal parts egg, butter, and onion that are blended together to form a cream. The cream may be packaged in a tube, similar to a toothpaste tube or small tub. The containers may be available in a variety of sizes. The cream may be applied to the affected area for three days until condition improves.

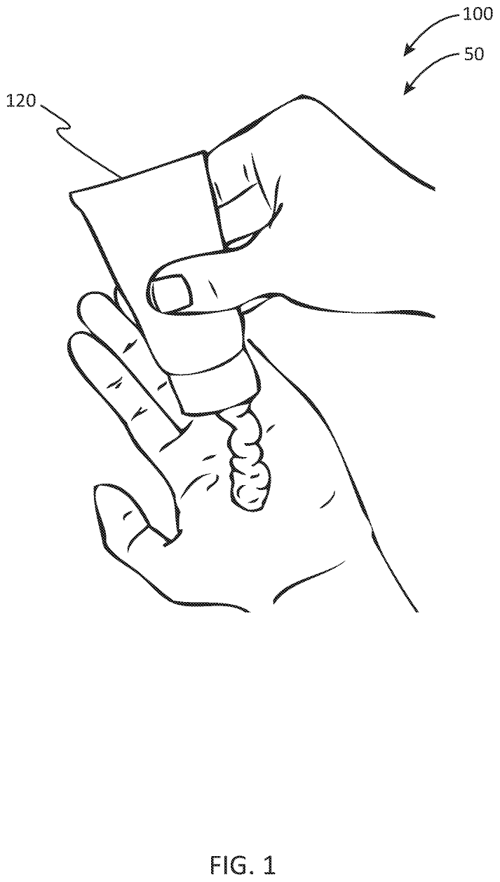

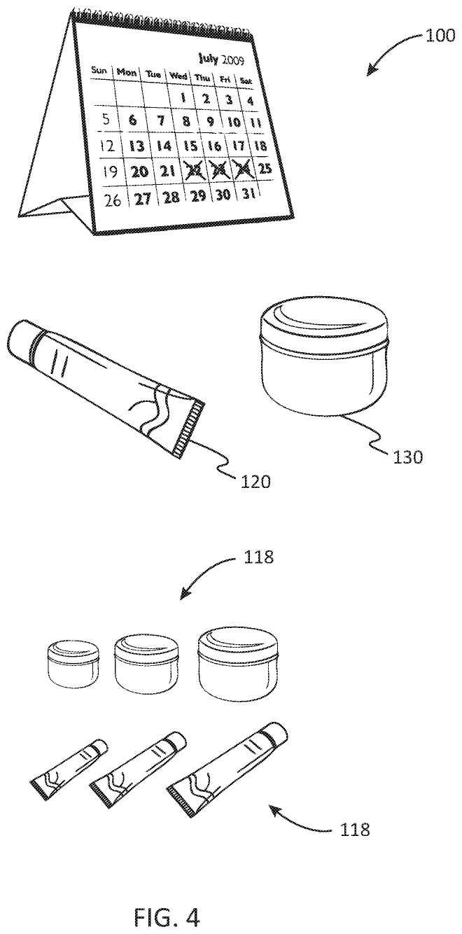

Referring now more specifically to the drawings by numerals of reference, there is shown in FIGS. 1-5, various views of a hemorrhoid treatment system 100.

FIG. 1 shows a hemorrhoid treatment system 100 during a ready for ‘in-use’ condition 50, the cream 110 being squeeze-dispensed from a deformable tube 120 (cap not shown). Here, the hemorrhoid treatment system 100 may be beneficial for use by a user to soothe hemorrhoids with treatment (application is onto the hemorrhoid). As illustrated, the hemorrhoid treatment system 100 may be used according to a method 500 comprising the steps at least of step one 501 providing a mixture of ingredients consisting of equal portions of egg; butter; and onion; and step 502 blending (during manufacture) the mixture to form a cream 110; the step 503 of applying the cream 110 topically to hemorrhoids to soothe the hemorrhoids in at least one or more applications. Those with ordinary skill in the art will now appreciate that upon reading this specification and by their understanding the art of treating hemorrhoids as described, methods of mixing, applying and removing will be understood by those knowledgeable in such art.

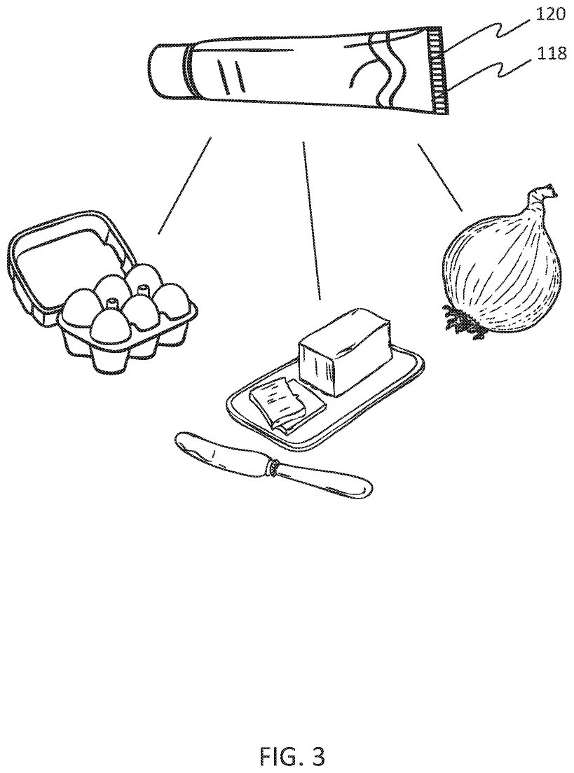

The hemorrhoid treatment system 100 comprises a mixture of ingredients formed as a cream 110 consisting essentially of equal portions of egg; butter; and onion; (FIG. 3) and a substantially air-tight container 118 (substantially air-tight when in a closed-state to avoid degradation of the product) that when in an open-state is suitable for removing (squeezing from a tube via deformable tube 120 or by hand from an openable container 130); the mixture in a cream form for application to hemorrhoids. The cream 110 is able to be applied (step 503) once dispensed from the substantially air-tight container 118. The mixture of ingredients is organic consisting of only organic ingredients and does not comprise dye(s), perfume(s), preservative(s), and stabilizer(s), as indicated in FIG. 3.

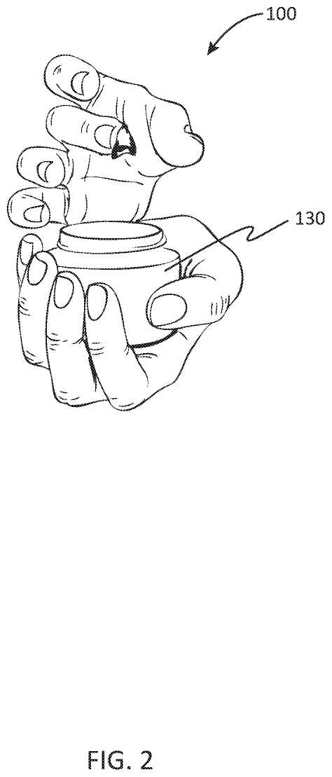

The applying 503 is preferably done over a three day period. The applying 503 may be done over at least a one day period for less severe cases or done over at least a three day period for severe cases or until a condition of the hemorrhoids improves to a ‘soothed state’. The at least one application of the cream 110 may comprise manually squeeze-dispensing of the cream 110 from a deformable tube 120 (FIG. 1). Alternately, the at least one application of the cream 110 may comprise manually hand-dispensing of the cream 110 from an openable container 130 (FIG. 2).

The mixture of ingredients is organic consisting of only organic ingredients; the mixture of ingredients does not comprise dye(s); perfume(s); preservative(s); or stabilizer(s).

A method of hemorrhoid treatment with the hemorrhoid treatment system comprises the steps of providing 501 a mixture of ingredients consisting essentially of equal portions of egg; butter; and onion, as a cream 110 when mixed; and blending 502 the mixture (during manufacture) to form the cream 110; and applying 503 the cream 110 topically to hemorrhoids to soothe the hemorrhoids in at least one or more application(s). The step of applying 503 is preferably done over a three day period as previously mentioned; wherein at least one application of the cream 110 may comprise manually squeeze-dispensing of the cream 110 from a deformable tube 120 (FIG. 1). Other dispensing means may be employed as are suitably equivalent; wherein the mixture of ingredients is organic consisting of only organic ingredients and does not comprise dye(s), perfume(s), preservative(s), and stabilizer(s). Upon reading this specification, it should be appreciated that, under appropriate circumstances, considering such issues as user preferences, design preference, structural requirements, marketing preferences, cost, available materials, technological advances, etc., other ingredient arrangements such as, for example, the addition of other organic ingredients may be added, etc., may be sufficient.

According to one embodiment, the hemorrhoid treatment system 100 may be arranged as a kit. In particular, the hemorrhoid treatment system 100 may further include a set of instructions. The instructions may detail functional relationships in relation to the structure of the hemorrhoid treatment system 100 such that the hemorrhoid treatment system 100 can be used, maintained, or the like, in a preferred manner.

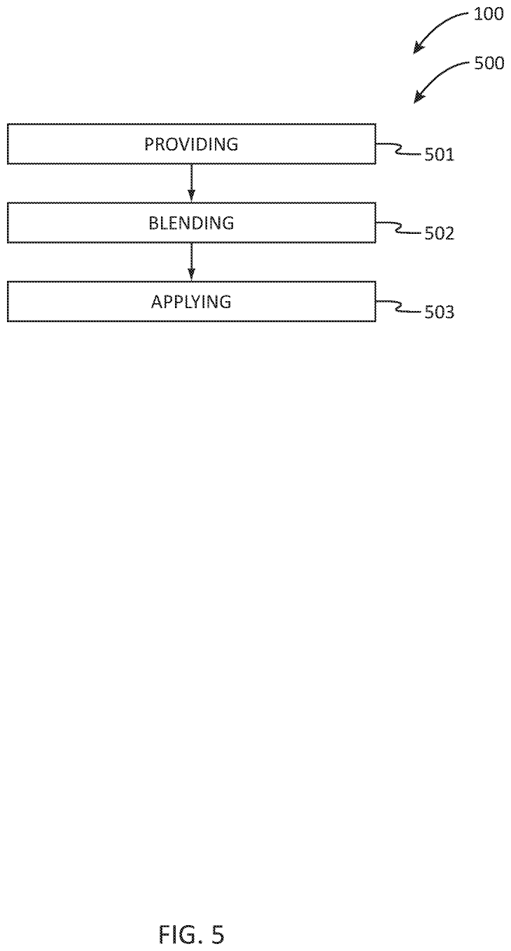

FIG. 5 shows the various sizes of packaging options of deformable tube 120 and openable container 130, both versions with lids on. It should also be noted that the steps described in the method of use 500 can be carried out in many different orders according to user preference. The use of “step of” should not be interpreted as “step for”, in the claims and is not intended to invoke the provisions of 35 U.S.C. § 112(f). It should also be noted that, under appropriate circumstances, considering such issues as design preference, user preferences, marketing preferences, cost, structural requirements, available materials, technological advances, etc., other methods for manufacture and use of hemorrhoid treatment system 100, are taught.

The embodiments of the invention described are exemplary and numerous modifications, variations and rearrangements can be readily envisioned to achieve substantially equivalent results, all of which are intended to be embraced within the spirit and scope of the invention. Further, the purpose of the foregoing abstract is to enable the U.S. Patent and Trademark Office and the public generally, and especially the scientist, engineers and practitioners in the art who are not familiar with patent or legal terms or phraseology, to determine quickly from a cursory inspection the nature and essence of the technical disclosure of the application.

### Exemplary Embodiments

1. A method of hemorrhoid treatment with a hemorrhoid treatment system comprising blending a mixture of ingredients consisting of equal portions of egg, butter, and onion into cream form; and packaging the cream into a package to facilitate topical application. 2. The method of embodiment 1, wherein topical application is application to hemorrhoids. 3. The method of embodiment 2, wherein the package is a deformable tube. 4. The method of embodiment 2, wherein the package is an openable container. 5. The method of embodiment 2, wherein each of the mixture's ingredients come from organically certified sources. 6. The method of embodiment 5, wherein the mixture of ingredients does not comprise dye(s). 7. The method of embodiment 6, wherein the mixture of ingredients does not comprise perfume(s). 8. The method of embodiment 7, wherein the mixture of ingredients does not comprise preservative(s). 9. The method of embodiment 8, wherein the mixture of ingredients does not comprise stabilizer(s).

10. The method of embodiment 9, wherein the mixture additionally contains natural or organically sourced dye(s). 11. The method of embodiment 10, wherein the mixture additionally contains natural or organically sourced perfume(s). 12. The method of embodiment 11, wherein the mixture additionally contains natural or organically sourced preservative(s). 13. The method of embodiment 12, wherein the mixture additionally contains natural or organically sourced stabilizer(s). 14. A method of making a hemorrhoid treatment system comprising providing egg, butter, and onion; emulsifying the egg, butter, and onion into a topical medicament; and packaging the cream into a package to facilitate topical application. 15. The method of embodiment 14, wherein topical application is application to hemorrhoids. 16. The method of embodiment 15, wherein the package is a deformable tube. 17. The method of embodiment 15, wherein the package is an openable container. 18. The method of embodiment 15, wherein each of the egg, butter, and onion ingredients come from organically certified sources. 19. The hemorrhoid treatment system of embodiment 15, wherein the mixture of ingredients is organic consisting of only organic said ingredients and does not comprise dye(s), perfume(s), preservative(s), and stabilizer(s). 20. A hemorrhoid treatment system to treat hemorrhoids, the system comprising a mixture of ingredients formed as a cream consisting essentially of equal portions of egg; butter; and onion; and a substantially air-tight container, when in a closed-state, that when in an open-state is suitable for removing said mixture in a cream form for application to hemorrhoids; wherein said cream is able to be applied once dispensed from said substantially air-tight container; and wherein the mixture of ingredients is organic consisting of only organic said ingredients and does not comprise dye(s), perfume(s), preservative(s), and stabilizer(s).

What is claimed is:

1. A method of hemorrhoid treatment with a hemorrhoid treatment system comprising:
blending a mixture of ingredients consisting essentially of equal portions of egg, butter, and onion into cream form;
and
packaging the cream into a package to facilitate topical application.

2. The method of claim 1, wherein topical application is application to hemorrhoids.

3. The method of claim 2, wherein the package is a deformable tube.

4. The method of claim 2, wherein the package is an openable container.

5. The method of claim 2, wherein each of the mixture's ingredients come from organically certified sources.

6. The method of claim 5, wherein the mixture of ingredients does not comprise dye.

7. The method of claim 6, wherein the mixture of ingredients does not comprise perfume.

8. The method of claim 7, wherein the mixture of ingredients does not comprise preservative.

9. The method of claim 8, wherein the mixture of ingredients does not comprise stabilizer.

10. The method of claim 9, wherein the mixture additionally contains natural or organically sourced dye(s).

11. The method of claim 10, wherein the mixture additionally contains natural or organically sourced perfume(s).

12. The method of claim 11, wherein the mixture additionally contains natural or organically sourced preservative(s).

13. The method of claim 12, wherein the mixture additionally contains natural or organically sourced stabilizer(s).

14. A method of making a hemorrhoid treatment system comprising:
providing egg, butter, and onion;
emulsifying the egg, butter, and onion into a topical medicament;
and
packaging the cream into a package to facilitate topical application.

15. The method of claim 14, wherein topical application is application to hemorrhoids.

16. The method of claim 15, wherein the package is a deformable tube.

17. The method of claim 15, wherein the package is an openable container.

18. The method of claim 15, wherein each of the egg, butter, and onion ingredients come from organically certified sources.

19. The hemorrhoid treatment system of claim 15F's, wherein the mixture of ingredients is organic consisting of only organic the ingredients and does not comprise dye(s), perfume(s), preservative(s), and stabilizer(s).

20. A hemorrhoid treatment system to treat hemorrhoids, the system comprising:
a mixture of ingredients formed as a cream consisting essentially of equal portions of
egg;
butter; and
onion;
and
a substantially air-tight container that when in an open-state allows removing the cream for application to hemorrhoids,
wherein the ingredients come from natural or certified organic sources.
